(19) United States
(12) Patent Application Publication
(10) Pub. No.: US 2012/0148664 A1
(43) Pub. Date: Jun. 14, 2012

(54) CHEMICAL MODIFICATIONS MOTIFS FOR MIRNA INHIBITORS AND MIMETICS

(75) Inventors: Christina Dalby, Boulder, CO (US); William S. Marshall, Boulder, CO (US)

(73) Assignee: Miragen Therapeutics, Boulder, CO (US)

(21) Appl. No.: 13/377,076

(22) PCT Filed: Jun. 8, 2010
(86) PCT No.: PCT/US10/37821

§ 371 Date: Mar. 2, 2012

Related U.S. Application Data
(60) Provisional application No. 61/185,033, filed on Jun. 8, 2009.

(51) Int. Cl. A61K 31/7088; A61K 9/127; A61P 9/00; A61P 9/04; A61P 9/14; A61K 9/00; C07H 21/00; A61P 9/10; B82Y 5/00

(52) U.S. Cl. 424/450; 536/24.5; 514/44 A; 424/400; 977/700; 977/915

(57) ABSTRACT

The present invention provides polynucleotides having chemistry patterns that provide for improved stability, potency, and/or toxicity relative to their use as miRNA inhibitors or miRNA mimetics. The invention further provides pharmaceutical compositions and formulations comprising the polynucleotides, and methods for treating patients having a condition associated with miRNA or mRNA expression.

### CROSS-REFERENCE TO RELATED APPLICATIONS

This application claims the benefit of priority of U.S. Provisional Application No. 61/185,033, filed Jun. 8, 2009, which is hereby incorporated by reference in its entirety.

### FIELD OF THE INVENTION

The present invention relates to chemical motifs for microRNA (miRNA or miR) inhibitors and mimetics, and particularly to chemically modified miRNA sense and antisense polynucleotides having advantages in potency, stability, and/or toxicity when administered to a patient.

### BACKGROUND

MicroRNAs (miRs) have been implicated in a number of biological processes including regulation and maintenance of cardiac function (see, Chien K R, Molecular Medicine: MicroRNAs and the tell-tale heart, Nature 447, 389-390 (2007)). Therefore, miRs represent a relatively new class of therapeutic targets for conditions such as cardiac hypertrophy, myocardial infarction, heart failure, vascular damage, and pathologic cardiac fibrosis, among others. miRs are small, non-protein coding RNAs of about 18 to about 25 nucleotides in length, and act as repressors of target mRNAs by promoting their degradation, when their sequences are perfectly complementary, or by inhibiting translation, when their sequences contain mismatches. The mature miRNA strand is incorporated into the RNA-induced silencing complex (RISC), where it associates with its target RNAs by base-pair complementarity.

miRNA function may be targeted therapeutically by antisense polynucleotides or by polynucleotides that mimic miRNA function (“miRNA mimetic”). However, when polynucleotides are introduced into intact cells they are attacked and degraded by nucleases leading to a loss of activity. While polynucleotide analogues have been prepared in an attempt to avoid their degradation, e.g. by means of 2′ substitutions (B. Sproat et al., Nucleic Acids Research 17 (1989), 3373-3386), the modifications often affect the polynucleotide's potency for its intended biological action. Such reduced potency, in each case, may be due to an inability of the modified polynucleotide to form a stable duplex with the target RNA and/or a loss of interaction with the cellular machinery.

Chemistry patterns or motifs for improving the stability, potency and/or toxicity profile of miRNA inhibitors and miRNA mimetics are needed for effectively targeting miRNA function in a therapeutic context.

### SUMMARY OF THE INVENTION

The present invention provides polynucleotides having chemistry patterns that provide for improved stability, potency, and/or toxicity relative to their use as miRNA inhibitors or miRNA mimetics. The invention further provides pharmaceutical compositions and formulations comprising the polynucleotides, and methods for treating patients having a condition associated with miRNA or mRNA expression.

In one aspect, the invention provides a polynucleotide having one or more nucleotide modifications at 2′ positions, and at least one terminal or “cap” modification. The chemical modification motif may obviate the need for fully phosphorothioate-linked polynucleotides and/or full length antisense or sense miRNA sequences. The polynucleotide is a miRNA inhibitor or miRNA mimetic, and as shown herein, provides an improved potency over unmodified polyribonucleotides, and/or over other potential polynucleotide modifications.

For example, the polynucleotide may be a miRNA inhibitor or miRNA mimetic having one or a combination of 2′ modifications as described herein, such as those selected from O-alkyl (e.g., 0-methyl or “OMe”), halo (e.g., fluoro), deoxy (H), and locked nucleic acid, and in some embodiments, substantially all, or all, nucleotide 2′ positions are modified. The terminal or cap modification may be, in some embodiments, a 5′ and/or 3′ phosphorothioate monophosphate and/or abasic moiety, or other cap structure as described herein. The polynucleotide need not be fully phosphorothioate linked, but where such linkages are present, such linkages may be placed, for example, between the two terminal nucleotides on the 5′ end and the two terminal nucleotides on the 3′ end. The nucleotide sequence may be full length relative to a mature miRNA or full length antisense miRNA (mature form), but in some embodiments the polynucleotide comprises a truncated miRNA sequence or a truncated miRNA antisense sequence. Such modified truncated sequences may show high levels of potency, even when compared with longer (unmodified or conventionally modified) counterparts. The polynucleotide may be an antagomir having an antisense sequence complementary to (all or portions of) miR-15b, miR-21, miR-208a, or others as described herein.

In a second aspect, the invention provides a pharmaceutical composition or formulation comprising the polynucleotide of the invention, and a pharmaceutically acceptable carrier. The pharmaceutical composition may be formulated in a variety of pharmaceutically-acceptable forms, including colloidal dispersion system, macromolecular complex, nanocapsule, microsphere, bead, oil-in-water emulsion, micelle, mixed micelle, or liposome. The composition may include conjugates with cholesterol and other molecules, such as targeting ligands, for delivering the polynucleotide into target mammalian cells.

In a third aspect, the invention provides a method for treating a patient having a condition associated with miRNA or mRNA expression. For example, the condition may be one or more of cardiac hypertrophy, myocardial infarction, heart failure, vascular damage, and pathologic cardiac fibrosis. Such conditions are treated, prevented or ameliorated by administering the polynucleotides and compositions of the invention. Thus, the invention provides a use of the modified polynucleotides and compositions of the invention for treatment of conditions associated with miRNA or mRNA expression.

### DESCRIPTION OF THE FIGURES

FIG. 1 is a Table showing exemplary miRNA modification patterns. The sequence shown is an antisense sequence (full length and truncated) for mature miR15b. A description of the abbreviations is provided in Table 3. The “alias” for each exemplified RNA includes: the miRNA target (e.g., 15b); the 2′ structure (O-methyl, “OMe”; or O-methyl and fluoro, “Me/F”; or O-methyl and deoxy, “Me/H”; or locked nucleic acid “LNA”); the size of the polynucleotide (FL for full length or 16-mer); and structure at the terminus or internal linkages including: PO for phosphodiester linked, PS for phosphorothioate-linked, PS_EO for every other phosphorothioate-linked. PS_EC for phosphorothioate end-cap, Abasic, and POS for 5′ and 3′ phosphorothioate monophosphate.

FIG. 2 shows the in vitro test results for the modified polynucleotides of FIG. 1 in HeLa cells at two concentrations, 10 nM (left bar in each set) and 0.1 nM (right bar in each set), using a dual-luciferase assay. The larger the value of the luciferase ratio, the better the potency of the inhibitor. The results are grouped by length, 16-mer and Full Length. The top performing chemistries are shown in Table 4.

FIG. 3 shows the results for phosphorothioate monophosphate modified polynucleotides in direct comparison to either phosphodiester or phosphorothioate backbone at 10 nM (left bar in set) and 0.1 nM (right bar in set) in the dual luciferase assay. PO is phosphodiester linked, PS is phosphorothioate linked, and PO_POS is phosphodiester linked with terminal phosphorothioate monophosphate on both the 3′ and the 5′ end.

FIG. 4 shows knockdown of miR-15b in mice with polynucleotides 5-14 from Table 5. miR-15b abundance in both the liver (left bar in set) and the heart (right bar in set) was determined and the data compared to saline injected mice.

FIG. 5 shows inhibition of miR-208a with modified antisense polynucleotides in neonatal rat cardiomyocytes. The results in FIG. 5 are quantitative PCR for βMHC expression. Left bar shows results at 100 nM inhibitor and right bar shows results at 1 nM inhibitor.

FIG. 6 shows inhibition of miR-21 by antisense polynucleotides with various modifications in the dual luciferase assay.

FIG. 7 shows the in vivo tissue distribution of four different modified miR-15b antisense polynucleotides following injection in mice at the indicated dosages.

### DETAILED DESCRIPTION OF THE INVENTION

The present invention provides polynucleotides having chemistry patterns that provide for improved stability, potency, and/or toxicity relative to their use as miRNA inhibitors or miRNA mimetics. The invention further provides pharmaceutical compositions and formulations comprising the polynucleotides, and methods for treating patients having a condition associated with miRNA or mRNA expression.

### Modified Polynucleotides

The polynucleotide has one or more nucleotide modifications at 2′ positions, and at least one terminal modification or “cap,” as described in detail below. The polynucleotide is a miRNA inhibitor or miRNA mimetic, and exhibits an improved potency over unmodified polyribonucleotides, and/or over other potential polynucleotide modifications.

As used herein, a “miRNA inhibitor” is a polynucleotide having a sequence that is antisense, either complementary or partially complementary as described herein, to a mature single-stranded miRNA or portion thereof. A “miRNA mimetic” is a polynucleotide having a sequence corresponding to (identical or substantially identical as described herein) to a mature single-stranded miRNA or portion thereof.

The polynucleotide has one or more nucleotide modifications (with respect to a 2′ hydroxyl) at 2′ positions. Incorporation of Z-modified nucleotides, in antisense oligonucleotides for example, may increase both resistance of the oligonucleotides to nucleases and their thermal stability with complementary RNA. Various modifications at the 2′ positions may be independently selected from those that provide increased nuclease sensitivity, without compromising molecular interactions with the RNA target or cellular machinery. Such modifications may be selected on the basis of their increased potency in vitro or in vivo. Exemplary methods for determining increased potency (e.g., IC50) for miRNA inhibition are described herein.

in some embodiments the 2′ modification may be independently selected from O-alkyl (which may be substituted), halo, deoxy (H), and locked nucleic acid. In certain embodiments, substantially all, or all, nucleotide 2′ positions are modified, e.g., as independently selected from O-alkyl (e.g., O-methyl), halo (e.g., fluoro), deoxy (H), and locked nucleic acid, For example, the 2′ modifications may each be independently selected from O-methyl and fluoro. In exemplary embodiments, purine nucleotides each have a 2′ OMe and pyrimidine nucleotides each have a 2′-F. In certain embodiments, from one to about five 2′ positions, or from about one to about three 2′ positions are left unmodified (e.g., as 2′ hydroxyls).

2′ modifications in accordance with the invention also include small hydrocarbon substituents. The hydrocarbon substituents include alkyl, alkenyl, alkynyl, and alkoxyalkyl, where the alkyl (including the alkyl portion of alkoxy), alkenyl and alkynyl may be substituted or unsubstituted. The alkyl, alkenyl, and alkynyl may be C1 to C10 alkyl, alkenyl or alkynyl, such as C2 or C3. The hydrocarbon substituents may include one or two or three non-carbon atoms, which may be independently selected from N, O, and/or S. The 2′ modifications may further include the alkyl, alkenyl, and alkynyl as O-alkyl, O-alkenyl, and O-alkynyl.

Exemplary 2′ modifications in accordance with the invention include 2′-O-alkyl (C1-3 alkyl, such as 2′OMe or 2′OEt), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) substitutions.

The 2′ modification may be ON/le on all nucleotide residues, or on all purine nucleotides.

In certain embodiments, the polynucleotide contains at least one Z-halo modification (e.g., in place of a 2′ hydroxyl), such as 2′-fluoro, 2′-chloro, 2′-bromo, and 2′-iodo. In some embodiments, the 2′ halo modification is fluoro. The polynucleotide may contain from 1 to about 20 2′-halo modifications (e.g., fluoro), or from 1 to about 10, or from 1 to about 5 2′-halo modifications (e.g., fluoro). In some embodiments, the polynucleotide contains all 2′-fluoro nucleotides, or 2′-fluoro on all pyrimidine nucleotides. In certain embodiments, the 2′-fluoro groups are independently di-, tri-, or un-methylated.

The polynucleotide may have one or more 2′-deoxy modification (e.g., H for 2′ hydroxyl), but may contain from 1 to about 20 2′-deoxy modifications, or from 1 to about 10, or from 1 to about 5 2′-deoxy modifications. In some embodiments, the polynucleotide contains all 2′-deoxy nucleotides.

In certain embodiments, the polynucleotide contains one or more “conformationally constrained” or bicyclic sugar nucleoside modifications (BSN) that confer enhanced thermal stability to complexes formed between the polynucleotide containing BSN and their complementary microRNA target strand. For example, in one embodiment, the polynucleotide contains one or more locked nucleic acid (LNAs) residues. LNAs are described, for example, in U.S. Pat. No. 6,268,490, U.S. Pat. No. 6,316,198, U.S. Pat. No. 6,403,566, U.S. Pat. No. 6,770,748, U.S. Pat. No. 6,998,484, U.S. Pat. No. 6,670,461, and U.S. Pat. No. 7,034,133, all of which are hereby incorporated by reference in their entireties. “Locked nucleic acids” (LNAs) are modified nucleotides or ribonucleotides that contain an extra bridge between the 2′ and 4′ carbons of the ribose sugar moiety resulting in a “locked” conformation. In one embodiment, the polynucleotide contains one or more LNAs having the structure shown in structure A. In another embodiment, the polynucleotide contains one or more LNAs having the structure shown in structure B. In yet another embodiment, the polynucleotide contains one or more LNAs having the structure shown in structure C.

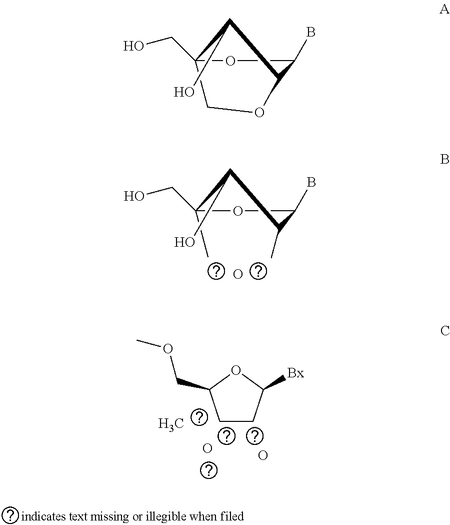

Other suitable BSN modifications that can be used in the polynucleotides of the invention include those described in U.S. Pat. No. 6,403,566 and U.S. Pat. No. 6,833,361, both of which are herein incorporated by reference in their entireties. In certain embodiments, the polynucleotide includes from about 1 to about 10 locked nucleic acids, or from 2 to about 5 locked nucleic acids.

In exemplary embodiments, the polynucleotide contains 2′ positions modified as 2′OMe. Alternatively, purine nucleotides are modified at the 2′ position as 2′OMe, with pyrimidine nucleotides modified at the 2′ position as 2′-fluoro.

The polynucleotide further comprises at least one terminal modification or “cap”. The cap may be a 5′ and/or a 3′-cap structure. The terms “cap” or “end-cap” include chemical modifications at either terminus of the polynucleotide (with respect to terminal ribonucleotides), and including modifications at the linkage between the last two nucleotides on the 5′ and and the last two nucleotides on the 3′ end. The cap structure as described herein increases resistance of the oligonucleotide to exonucleases without compromising molecular interactions with the RNA target or cellular machinery. Such modifications may be selected on the basis of their increased potency in vitro or in vivo. Exemplary methods for determining increased potency (e.g., IC50) for miRNA inhibition are described herein.

The cap can be present at the 5′-terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic mioety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In certain embodiments, the polynucleotide has, in addition to one or more 2′ modifications as described above, at least one terminal phosphorothioate monophosphate. The phosphorothioate monophosphate may support a higher potency of miRNA inhibitors and miRNA mimetics by inhibiting the action of exonucleases, and in some embodiments, obviates the need for fully phosphorotioate linked polynucleotides and/or full length inhibitors. The phosphorothioate monophosphate may be at the 5′ and/or 3′ end of the oligonucleotide. A phosphorothioate monophosphate is defined by the following structures, where B is base, and R is a 2′ modification as described above:

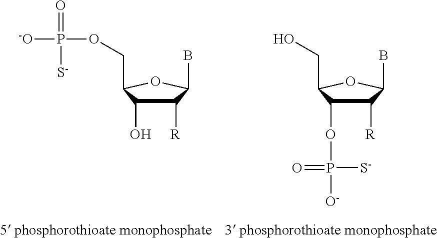

In certain embodiments, in addition to a phosphorothioate monophosphate at the 5′ and/or 3′ end, the polynucleotide contains all 2′ positions modified as 2′OMe, or alternatively, purine nucleotides are modified at the 2′ position as 2′OMe with pyrimidine nucleotides modified at the 2′ position as 2′-fluoro. As exemplified herein for miR-15b inhibitors, the poylnucleotide in these embodiments need not be fully phoshphorothioate-linked and/or need not be full length (with respect to the corresponding mature miRNA sequence). Phosphorothioate linkages may be present in some embodiments, such as between the last two nucleotides on the 5′ and the 3′ end (e.g., as part of a cap structure), or as alternating with phosphodiester bonds.

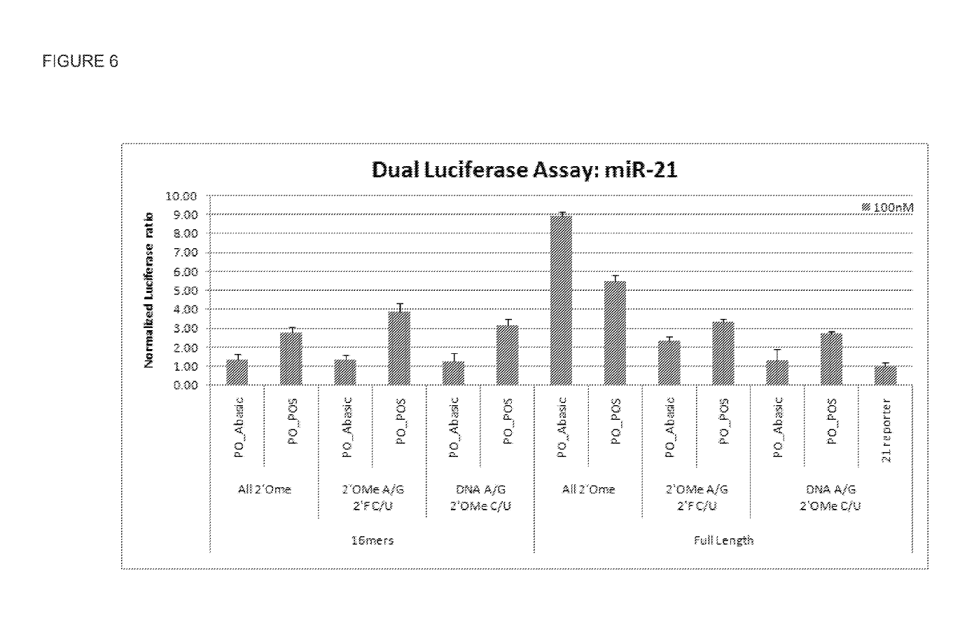

In these or other embodiments, the polynucleotide may contain at least one terminal abasic residue at either or both the 5′ and 3′ ends. An abasic moiety does not contain a commonly recognized purine or pyrimidine nucleotide base, such as adenosine, guanine, cytosine, uracil or thymine. Thus, such abasic moieties lack a nucleotide base or have other non-nucleotide base chemical groups at the 1′ position. For example, the abasic nucleotide may be a reverse abasic nucleotide, e.g., where a reverse abasic phosphoramidite is coupled via a 5′ amidite (instead of 3′ amidite) resulting in a 5′-5′ phosphate bond. The structure of a reverse abasic nucleoside for the 5′ and the 3′ end of a polynucleotide is shown below. Polynucleotides having such abasic cap structures with 2′OMe modifications may be particularly effective, as shown herein for miR-21 (FIG. 6)

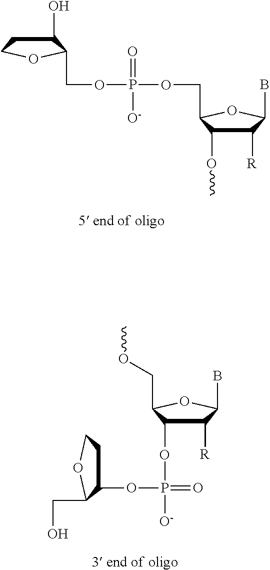

Phosphorothioate linkages have been used to render polynucleotides more resistant to nuclease cleavage. While the chemical modification patterns disclosed herein can accommodate phosphorothioate linkages (including as a cap structure as described), in certain embodiments, internal phosphorothioate linkages are rendered unnecessary by the 2′-modification and cap modification described. Nevertheless, in certain embodiments, the polynucleotide contains one or more internal phosphorothioate linkages (other than in the cap). For example, the polynucleotide may be partially phosphorothioate-linked, for example, phosphorothioate linkages may alternate with phophodiester linkages.

The polynucleotide may comprise, consist essentially of, or consist of, a full length or truncated miRNA sequence or a full length or truncated miRNA antisense sequence. As used herein, the term “full length” in reference to a miRNA sequence refers to the length of the mature miRNA sequence or its antisense counterpart. Thus, the inhibitors and mimetics described herein may be truncated or full-length (sense or antisense) mature miRNA sequences or may comprise these sequences in combination with other polynucleotide sequences. For example, the inhibitors and mimetics may, in some embodiments, correspond to pre- and pri-miRNA sequences or portions thereof, or may comprise other non-miRNA sequences. In certain embodiments, the chemical modification motif described herein renders full length antisense or sense miRNA (mature) sequences unnecessary.

The polynucleotide in certain embodiments is from 5 to 25 nucleotides in length, from 8 to 18 nucleotides in length, or from 12 to 16 nucleotides in length. In certain embodiments, the polynucleotide is about 8 nucleotides or less, about 10 nucleotides or less, about 12 nucleotides or less, or about 16 nucleotides or less in length. The polynucleotide in some embodiments is about 16 nucleotides in length.

The polynucleotide may have a nucleotide sequence designed to mimic or target a mature miRNA, such as a mature miRNA listed in Table 1 below. The polynucleotide may in these or other embodiments, also or alternatively be designed to target the pre- or pri-miRNA forms. In certain embodiments, the polynucleotide designed to inhibit a miRNA may have a sequence containing from 1 to 5 (e.g., 2, 3, or 4) mismatches relative to the fully complementary miRNA sequence (shown in Table 1 below). In other embodiments, the polynucleotide designed to mimic a miRNA may have a sequence containing from 1 to 5 (e.g., 2, 3, or 4) nucleotide substitutions relative to the mature miRNA sequence (shown in Table 1 below). Such antisense and sense sequences may be incorporated into shRNAs or other RNA structures containing stem and loop portions, for example. Such sequences are useful for, among other things, mimicking or targeting miRNA function for treatment or ameliorating cardiac hypertrophy, myocardial infarction, heart failure (e.g., congestive heart failure), vascular damage, and/or pathologic cardiac fibrosis, among others. Exemplary miRNA therapeutic utilities are disclosed in the US and PCT patent references listed in Table 1 below, each of which is hereby incorporated by reference in its entirety. The mature and pre-processed forms of miRNAs are disclosed in the patent references listed below, and such descriptions are also hereby incorporated by reference.

TABLE 1

| miRNA | miRNA Sequence | Reference |
|---|---|---|
| 1 | UGGAAUGUAAAGAAGUAUGUAU | WO 2009/012,468 |
| 100 | AACCCGUAGAUCCGAACUUGUG | WO 2009/012,468 |
| 10b | UACCCUGUAGAACCGAAUUUGUG | WO 2009/012,468 |
| 125b | UCCCUGAGACCCUAACUUGUGA | WO 2009/012,468 |
| 128 | UCACAGUGAACCGGUCUCUUU | WO 2007/070,483 |
| 133a | UUUGGUCCCCUUCAACCAGCUG | WO 2009/012,468 |
| 133b | UUUGGUCCCCUUCAACCAGCUA | WO 2009/012,468 |
| 139 | UCUACAGUGCACGUGUCUCCAG | WO 2009/012,468 |
| 143 | UGAGAUGAAGCACUGUAGCUC | WO 2007/070,483 |
| 145 | GUCCAGUUUUCCCAGGAAUCCCU | WO 2007/070,483 |
| 150 | UCUCCCAACCCUUGUACCAGUG | WO 2009/012,468 |
| 15a | UAGCAGCACAUAAUGGUUUGUG | WO 2009/062,169 |
| 15b | UAGCAGCACAUCAUGGUUUACA | WO 2009/062,169 |
| 16 | UAGCAGCACGUAAAUAUUGGCG | WO 2009/062,169 |
| 181b | AACAUUCAUUGCUGUCGGUGGGU | WO 2009/012,468 |
| 195 | UAGCAGCACAGAAAUAUUGGC | WO 2009/012,468 |
| 197 | UUCACCACCUUCUCCACCCAGC | WO 2009/012,468 |
| 199a | CCCAGUGUUCAGACUACCUGUUC | WO 2009/012,468 |
| 199b | miR-199b-5p | US 61/047,005 |
| | CCCAGUGUUUAGACUAUCUGUUC | |
| | miR-199b-3p | |
| | ACAGUAGUCUGCACAUUGGUUA | |
| 206 | UGGAAUGUAAGGAAGUGUGUGG | WO 2007/070,483 |
| 208a | AUAAGACGAGCAAAAAGCUUGU | WO 2008/016,924 |
| 208b | AUAAGACGAACAAAAGGUUUGU | WO 2009/018,492 |
| 20a | UAAAGUGCUUAUAGUGCAGGUAG | US 60/950,565 |
| 21 | UAGCUUAUCAGACUGAUGUUGA | WO 2009/058,818 |
| 214 | ACAGCAGGCACAGACAGGCAGU | US 61/047,005 |
| 22 | AAGCUGCCAGUUGAAGAACUGU | WO 2009/012,468 |
| 221 | AGCUACAUUGUCUGCUGGGUUUC | WO 2009/012,468 |
| 222 | AGCUACAUCUGGCUACUGGGU | WO 2009/012,468 |
| 224 | CAAGUCACUAGUGGUUCCGUU | WO 2009/012,468 |
| 23a | AUCACAUUGCCAGGGAUUUCC | WO 2009/012,468 |
| 26a | UUCAAGUAAUCCAGGAUAGGCU | WO 2007/070,483 |
| 26b | UUCAAGUAAUUCAGGAUAGGU | WO 2009/012,468 |
| 28 | AAGGAGCUCACAGUCUAUUGAG | WO 2009/012,468 |
| 29a | UAGCACCAUCUGAAAUCGGUUA | WO 2009/018,493 |
| 29b | UAGCACCAUUUGAAAUCAGUGUU | WO 2009/018,493 |
| 29c | UAGCACCAUUUGAAAUCGGUUA | WO 2009/018,493 |
| 30a | UGUAAACAUCCUCGACUGGAAG | PCT/US2010/031,147 |
| 30b | UGUAAACAUCCUACACUCAGCU | PCT/US2010/031,147 |
| 30c | UGUAAACAUCCUACACUCUCAGC | WO 2009/012,468 |
| 30d | UGUAAACAUCCCCGACUGGAAG | PCT/US2010/031,147 |
| 30e | UGUAAACAUCCUUGACUGGAAG | PCT/US2010/031,147 |
| 342-3p | UCUCACACAGAAAUCGCACCCGU | WO 2009/012,468 |
| 382 | GAAGUUGUUCGUGGUGGAUUCG | WO 2009/012,468 |
| 422a | ACUGGACUUAGGGUCAGAAGGC | US 2009/0,226,375 |
| 378 | ACUGGACUUGGAGUCAGAAGG | WO 2009/012,468 |
| 424 | CAGCAGCAAUUCAUGUUUUGAA | WO 2009/062,169 |
| 483-3p | UCACUCCUCUCCUCCCGUCUU | WO 2009/012,468 |
| 484 | UCAGGCUCAGUCCCCUCCCGAU | WO 2009/012,468 |
| 486-5p | UCCUGUACUGAGCUGCCCCGAG | WO 2009/012,468 |
| 497 | CAGCAGCACACUGUGGUUUGU | WO 2009/062,169 |
| 499 | UUAAGACUUGCAGUGAUGUUU | WO 2009/018,492 |
| 542-5p | UCGGGGAUCAUCAUGUCACGAGA | WO 2009/012,468 |
| 92a | UAUUGCACUUGUCCCGGCCUGU | WO 2009/012,468 |
| 92b | UAUUGCACUCGUCCCGGCCUCC | WO 2009/012,468 |
| let-7a | UGAGGUAGUAGGUUGUAUAGUU | WO 2009/012,468 |
| let-7b | UGAGGUAGUAGGUUGUGUGGUU | WO 2009/012,468 |
| let-7c | UGAGGUAGUAGGUUGUAUGGUU | WO 2009/012,468 |
| let-7d | AGAGGUAGUAGGUUGCAUAGUU | WO 2009/012,468 |
| let-7e | UGAGGUAGGAGGUUGUAUAGUU | WO 2009/012,468 |
| let-7f | UGAGGUAGUAGAUUGUAUAGUU | WO 2009/012,468 |
| let-7g | UGAGGUAGUAGUUUGUACAGUU | WO 2009/012,468 |
| 451 | AAACCGUUACCAUUACUGAGUU | PCT/US2010/034,227 |

In certain embodiments, the polynucleotide comprises an antisense sequence fully or partially complementary (as described) to all or a portion of pri, pre-, or mature miR-15b, miR-208a, or miR-21.

miR-15b, including its structure and processing, and its potential for treating cardiac hypertrophy, heart failure, or myocardial infarction (among others), are described in WO 20091062169, which is hereby incorporated by reference in its entirety. The pre-miRNA sequence for human miR-15b, which may be used for designing inhibitory miRNAs in accordance with the invention, is (from 5′ to 3′):

UUGAGGCCUU AAAGUACUGU AGCAGCACAU CAUGGUUUAC
AUGCUACAGU CAAGAUGCGA AUCAUUAUUU GCUGCUCUAG
AAAUUUAAGG AAAUUCAU.

miR-208a, including its structure and processing, and its potential for treating cardiac hypertrophy, heart failure, or myocardial infarction (among others), are described in WO 2009/018492, which is hereby incorporated by reference in its entirety. The pre-miRNA sequence for human miR-208a, which may be used for designing inhibitory miRNAs in accordance with the invention, is (from 5′ to 3′):

ACGGGCGAGC UUUUGGCCCG GGUUAUACCU GAUGCUCACG
UAUAAGACGA GCAAAAAGCU TGUUGGUCAG A.

miR-21, including its structure and processing, and its potential for treating cardiac hypertrophy, heart failure, or myocardial infarction (among others), are described in WO 2009/058818, which is hereby incorporated by reference in its entirety. The pre-miRNA sequence for human miR-21, which may be used for designing inhibitory miRNAs in accordance with the invention, is (from 5′ to 3′):

UGUCGGGUAG CUUAUCAGAC UGAUGUUGAC UGUUGAAUCU
CAUGGCAACA CCAGUCGAUG GGCUGUCUGA CA.

Where the target miRNA is miR-15b, miR-208a or miR-21, the polynucleotide may contain all 2′OMe or 2′OMe and 2′-F as described, and may contain phosphorothioate monophosphate caps at the 5′ and 3′ ends, and/or abasic residues at the 5′ and/or 3′ ends, and/or end-capped with phosphorothioate linkages. The polynucleotide may be partially phosphorothioate linked, or entirely phosphodiester linked other than optionally having phosphorothioate and caps. The antisense polynucleotide may be fully complementary to a truncated mature miRNA sequence, such as about 8, about 10, about 12, about 14, about 15, about 16, about 17, or about 18 nucleotides in length (e.g., about 14 to about 18 nucleotides in length). In some embodiments, the polynucleotide comprises or consists of (or consists essentially of) a full-length antisense sequence (relative to the mature miRNA). In this context, the term “consists essentially of” means that additional nucleotides may be added to the 5′ end and/or 3′ end, such as from 1 to 3 nucleotides on each end, so long as the potency and/or specificity of the polynucleotide for its target are not affected.

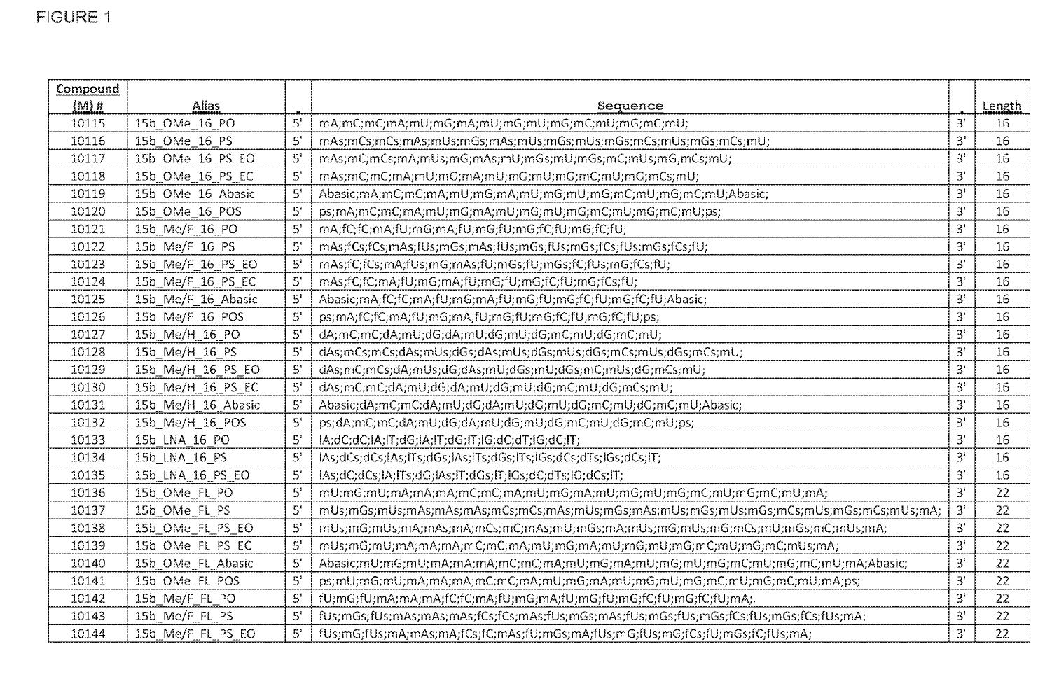

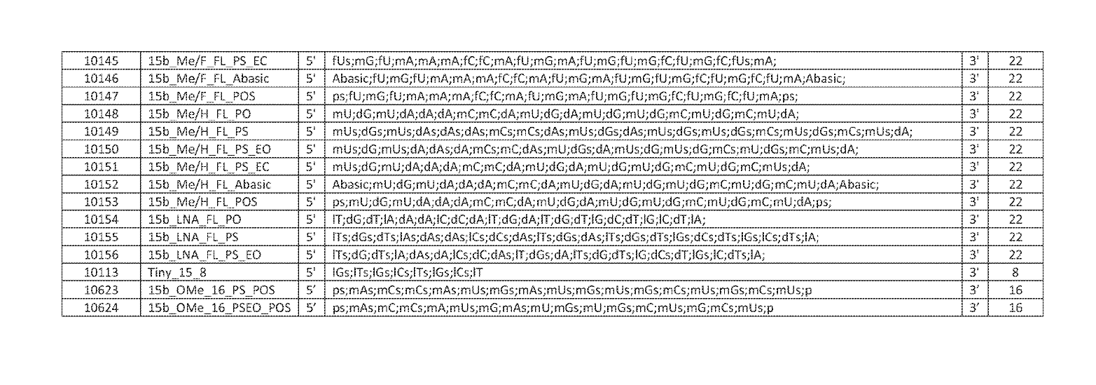

The polynucleotide may have a sequence/structure selected from FIG. 1, or Table 2 below. Abbreviations are shown in Table 3.

TABLE 2

| miRNA | | | |
|---|---|---|---|
| target | Alias | Sequence (5′ to 3′) | Length |
| 15b | 15b_OMe_16_POS | ps-mAmCmCmAmUmGmAmUmGmUmGmCmUmGmCmU-ps | 16 |
| 15b | 15b__Me/F_16_POS | ps-mAfCfCmAfUmGmAfUmGfUmGfCfUmGfCfU-ps | 16 |
| 15b | 15b__OMe_FL_POS | ps-mUmGmUmAmAmAmCmCmAmUmGmAmUmGmUmGmCmUmGmCmUmA-ps | 22 |
| 15b | 15b_Me/F_FL_POS | ps-fUmGfUmAmAmAfCfCmAfUmGmAfUmGmmGfCfUmGfCfUmA-ps | 22 |
| 208 | 208a_OMe_16_POS | ps-mCmUmUmUmUmUmGmCmUmCmGmUmCmUmUmA-ps | 16 |
| 208 | 208a_Me/F_16_POS | ps-fCfUfUfUfUfUmGfCfUfCmGfUfCfUfUmA-ps | 16 |
| 208 | 208a_OMe_FL_POS | ps-mAmCmAmAmGmCmUmUmUmUmUmGmCmUmCmGmUmCmUmUmAmU-ps | 22 |
| 208 | 208a_OMe/F_FL_POS | ps-mAfCmAmAmGfCfUfUfUmGfCfUfCmGfUfCfUfUmAfU-ps | 22 |
| 21 | 21_OMe_16_POS | ps-mAmUmCmAmGmUmCmUmGmAmUmAmAmGmCmU-ps | 16 |
| 21 | 21_Me/F_16_POS | ps-mAfUfCmAmGfUfCfUmGmAfUmAmAmGfCfU-pa | 16 |
| 21 | 21_OMe_FL_POS | ps-mUmCmAmAmCmAmUmCmAmGmUmCmUmGmAmUmAmAmGmCmUmA-ps | 22 |
| 21 | 21_Me/F_FL_POS | ps-mFfCmAmSfCmAmFfCmAmGmFfCmFmGmAmFmAmAmGfCmFmA-ps | 22 |

The synthesis of polynucleotides, including modified polynucleotides, by solid phase synthesis is well known and is reviewed in New Chemical Methods for Synthesizing Polynucleotides. Caruthers M H, Beaucage S L, Efcavitch J W, Fisher E F, Matteucci M D, Stabinsky Y. Nucleic Acids Symp. Ser. 1980;(7):215-23.

### Compositions, Formulations, and Delivery

The polynucleotide may be incorporated within a variety of macromolecular assemblies or compositions. Such complexes for delivery may include a variety of liposomes, nanoparticles, and micelles, formulated for delivery to a patient. The complexes may include one or more fusogenic or lipophilic molecules to initiate cellular membrane penetration. Such molecules are described, for example, in U.S. Pat. No. 7,404,969 and U.S. Pat. No. 7,202,227, which are hereby incorporated by reference in their entireties.

The composition or formulation may employ a plurality of therapeutic polynucleotides, each independently as described herein. For example, the composition or formulation may employ from 1 to 5 miRNA inhibitors and/or miRNA mimetics, each independently as above, e.g., with reference to Tables 1, 2 and FIG. 1.

The polynucleotides of the invention may be formulated as a variety of pharmaceutical compositions. Pharmaceutical compositions will be prepared in a form appropriate for the intended application. Generally, this will entail preparing compositions that are essentially free of pyrogens, as well as other impurities that could be harmful to humans or animals. Exemplary delivery/formulation systems include colloidal dispersion systems, macromolecule complexes, nanocapsules, microspheres, beads, and lipid-based systems including oil-in-water emulsions, micelles, mixed micelles, and liposomes. Commercially available fat emulsions that are suitable for delivering the nucleic acids of the invention to cardiac and skeletal muscle tissues include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO03/093449, which are hereby incorporated by reference in theft entireties.

The pharmaceutical compositions and formulations may employ appropriate salts and buffers to render delivery vehicles stable and allow for uptake by target cells. Aqueous compositions of the present invention comprise an effective amount of the delivery vehicle comprising the inhibitor polynucleotides or miRNA polynucleotide sequences (e.g. liposomes or other complexes), dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” may include one or more solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Supplementary active ingredients also can be incorporated into the compositions.

Administration or delivery of the pharmaceutical compositions according to the present invention may be via any route so long as the target tissue is available via that route. For example, administration may be by intradermal, subcutaneous, intramuscular, intraperitoneal or intravenous injection, or by direct injection into target tissue (e.g., cardiac tissue). Pharmaceutical compositions comprising miRNA inhibitors or expression constructs comprising miRNA sequences may also be administered by catheter systems or systems that isolate coronary circulation for delivering therapeutic agents to the heart. Various catheter systems for delivering therapeutic agents to the heart and coronary vasculature are known in the art. Some non-limiting examples of catheter-based delivery methods or coronary isolation methods suitable for use in the present invention are disclosed in U.S. Pat. No. 6,416,510; U.S. Pat. No. 6,716,196; U.S. Pat. No. 6,953,466, WO 2005/082440, WO 2006/089340, U.S. Patent Publication No. 2007/0203445, U.S. Patent Publication No. 2006/0148742, and U.S. Patent Publication No. 2007/0060907, which are all hereby incorporated by reference in their entireties.

The compositions or formulations may also be administered parenterally or intraperitoneally. By way of illustration, solutions of the conjugates as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the conjugates in an appropriate amount into a solvent along with any other ingredients (for example as enumerated above) as desired. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject, Moreover, for human administration, preparations should meet sterility, pyrogenicity, general safety and purity standards as required by FDA Office of Biologics standards.

### Methods of Treatment

The invention provides a method for delivering polynucleotides to a mammalian cell, and methods for treating, ameliorating, or preventing the progression of a condition in a mammalian patient. The method generally comprises administering the polynucleotide or composition comprising the same to a mammalian patient. The polynucleotide, as already described, may be a miRNA inhibitor or a miRNA mimetic (e.g., having a nucleotide sequence designed to inhibit expression or activity of a miRNA). Thus, the patient may have a condition associated with RNA expression, such as miRNA expression, Such conditions include, for example, cardiac hypertrophy, myocardial infarction, heart failure (e.g., congestive heart failure), vascular damage, restenosis, or pathologic cardiac fibrosis. Thus, the invention provides a use of the modified polynucleotides and compositions of the invention for treating such conditions, and for the preparation of medicaments for such treatments as described.

miRNAs involved in conditions such as cardiac hypertrophy, myocardial infarction, heart failure (e.g., congestive heart failure), vascular damage, restenosis, and/or pathologic cardiac fibrosis, as well as sequences for targeting miRNA function are described in WO 2008/016924, WO 2009/058818, WO 2009/018492, WO 2009/018493, WO 2009/012468, WO 2009/062169, and WO 2007/070483, which are each hereby incorporated by reference in their entireties. Such miRNAs and sequences are further listed in Table 1, and modified polynucleotides based upon these sequences are shown in Table 2 and FIG. 1, and described herein.

In certain embodiments, the patient has one or more risk factors including, for example, long standing uncontrolled hypertension, uncorrected valvular disease, chronic angina, recent myocardial infarction, congenital predisposition to heart disease and pathological hypertrophy. Alternatively or in addition, the patient may have been diagnosed as having a genetic predisposition to, for example, cardiac hypertrophy, or may have a familial history of, for example, cardiac hypertrophy.

In this aspect, the present invention may provide for an improved exercise tolerance, reduced hospitalization, better quality of life, decreased morbidity, and/or decreased mortality in a patient with heart failure or cardiac hypertrophy.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

A panel of miRNA inhibitors (single stranded oligonucleotides) were synthesized targeting the miRNA miR-15b. The sequences and modification patterns are shown in Table 3 below, with abbreviations.

TABLE 3
List of molecules synthesized for chemistry optimization screen

| Nucleotide unit or | | Nucleotide unit or | |
|---|---|---|---|
| modification | Abbreviation | modification | Abbreviation |
| ribo A | rA | ribo A P═S | rAs |
| ribo G | rG | ribo G P═S | rGs |
| ribo C | rC | ribo C P═S | rCs |
| ribo U | rU | ribo U P═S | rUs |
| O-methyl A | mA | O-methyl A P═S | mAs |
| O-methyl G | mG | O-methyl G P═S | mGs |
| O-methyl C | mC | O-methyl C P═S | mCs |
| O-methyl U | mU | O-methyl U P═S | mUs |
| fluoro C | fC | fluoro C P═S | fCs |
| fluoro U | fU | fluoro U P═S | fUs |
| deoxy A | dA | deoxy A P═S | dAs |
| deoxy G | dG | deoxy G P═S | dGs |
| deoxy C | dC | deoxy C P═S | dCs |
| deoxy T | dT | deoxy T P═S | dTs |
| LNA A | IA | LNA A P═S | IAs |
| LNA G | IG | LNA G P═S | IGs |
| LNA C | IC | LNA C P═S | ICs |
| LNA T | IT | LNA T P═S | ITs |
| phosphate | p | | |
| phosphorothioate | ps | | |
| monophosphate | | | |

Alias Key

| | |
|---|---|
| Full Length | FL |
| Every other PS | EO |
| linkage | |
| phosphorothioate | PS |
| linked | |
| End capped with PS | EC |
| linkage | |
| 5′ and 3′ | POS |
| phosphorothioate | |
| monophosphate | |
| Phosphodiester | PO |
| linked | |

Three different lengths of reverse complement RNA inhibitors were synthesized against mature miR15-b, 8nt, 16nt and full length (22nt). Chemical modifications in this example included, 2′-OMe, 2′-F, 2′-deoxy, phosphorothioate linked, and LNA, which were combined in specific motifs. Motifs included phosphorothioate linkages between the two bases on either side (phosphorothioate end-capped). Additional modifications included end-caps with abasic (reverse abasic motif having a 5′-5′ phosphate bond on the 5′ end and/or 3′-3′ phosphate bond at the 3′ end as described herein) or phosphorothioate monophosphates to both the 3′ and the 5′ ends.

The structures of the polynucleotides synthesized are shown in FIG. 1.

### Example 1

### Inhibition of miR 15-b In Vitro

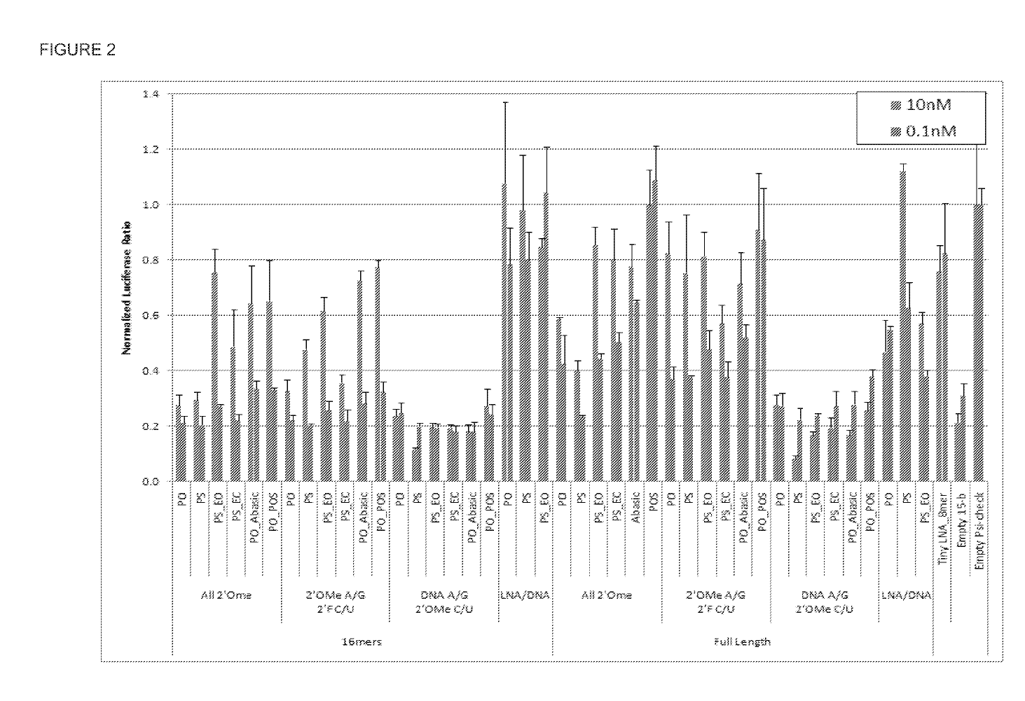

The panel was tested in HeLa cells at two concentrations, 10 nM and 0.1 nM. The readout was a dual-luciferase assay. This assay does not test the inhibition of the miRNA directly, but rather the effect of the inhibited miRNA which is shown as an increase in renilla luciferase. The 2nd luciferase, firefly, is not effected by inhibition of the miRNA and is used as an internal control. The larger the value of the luciferase ratio, the better the potency of the inhibitor. See, Vermeulen A, at al., Double-stranded regions are essential design components of potent inhibitors of RISC function RNA 13:723-730 (2007). The results of the screen are shown in FIG. 2.

FIG. 2 provides the results grouped by length—16 mer and Full Length. One noticeable chemical motif was the 2′0Me with phosphorothioate monophosphate.

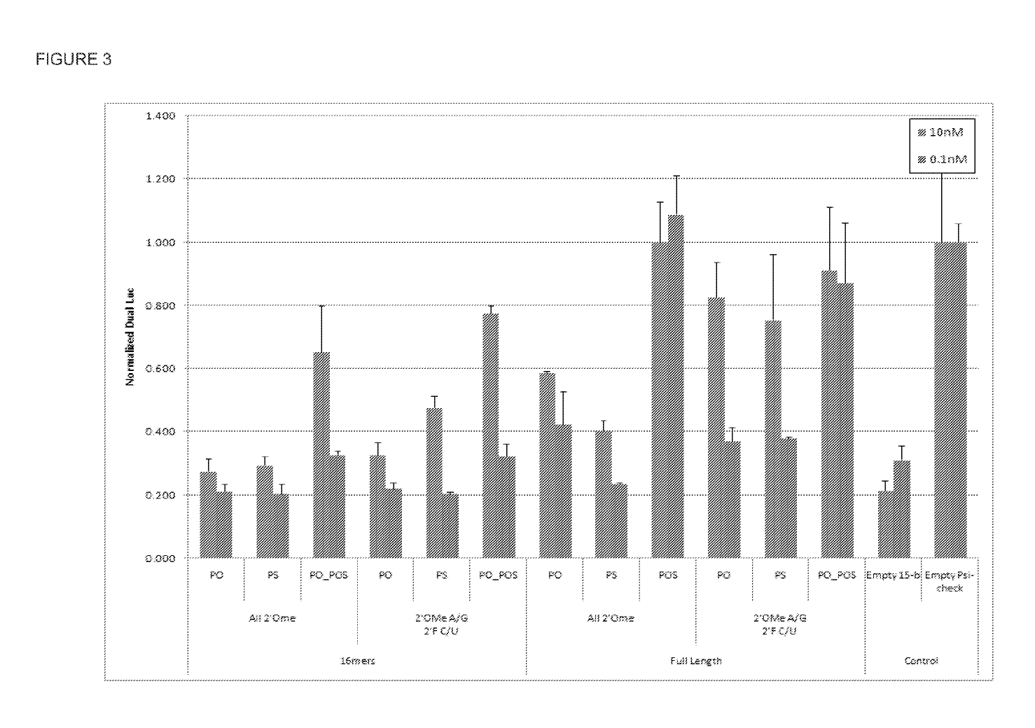

FIG. 3 highlights the phosphorothioate monophosphate in direct comparison to either phosphodiester or phosphorothioate backbone. For the “full length” inhibitors, it is equivalent at the 10 nm concentration to the comparators, but at the 0.1 nM concentration it is clearly much more potent. For the 16 mer length, the inhibitors without the phosphorothioate monophosphate do not show much activity at all. It is also striking that the fully phosphorothioate molecule is not as potent; therefore this end-capping method appears to be a significant contributor to potency.

The top fourteen performing inhibitors from the screen were chosen for 1050 determination, and these are listed in Table 4.

TABLE 4
Top Performing Chemistries

15b_OMe_16_Abasic
15b_OMe_16_POS
15b_Me/F_16_POS
15b_LNA_16_PO
15b_LNA_16_PS
15b_LNA_16_PS_EO
15b_OMe_FL_PS_EC
15b_OMe_FL_Abasic
15b_OMe_FL_POS
15b_Me/F_FL_PO
15b_Me/F_FL_PS_EO
15b_Me/F_FL_Abasic
15b_Me/F_FL_POS
Tiny_15_8

The molecules were transfected at six concentrations into HeLa cells ranging from 100 nM to 1 pM. After 48 hours, total RNA was purified and quantitative PCR was performed to measure levels of miR-15b and a control RNA. IC50s were calculated and are shown in the table below. The molecules containing the terminal phosphorothioate monophosphates are listed in bold in Table 5.

TABLE 5

| | | IC50 | Error by |
|---|---|---|---|
| | | (nM) | mean square |
| 1 | Tiny_15_8 | 2.28 | 0.0209 |
| 2 | 15b_LNA_16_PS | 0.00 | 0.0001 |
| 3 | 15b_LNA_16_PS_EO | 0.00 | 0.0002 |
| 4 | 15b_LNA_16_PO | 0.12 | 0.0004 |
| 5 | 15b_OMe_16_Abasic | 0.17 | 0.0159 |
| 6 | 15b_{—}OMe_{—}16_{—}POS | 0.08 | 0.0100 |
| 7 | 15b_{—}Me/F_{—}16_{—}POS | 1.04 | 0.0333 |
| 8 | 15b_OMe_FL_Abasic | 0.06 | 0.0037 |
| 9 | 15b_{—}OMe_{—}FL_{—}POS | 0.01 | 0.0036 |
| 10 | 15b_Me/F_FL_PO | 0.13 | 0.0125 |
| 11 | 15b_{—}Me/F_{—}FL_{—}POS | 0.18 | 0.0051 |
| 12 | 15b_Me/F_FL_Abasic | 0.86 | 0.0090 |
| 13 | 15b_Me/F_FL_PS_EO | 0.03 | 0.0022 |
| 14 | 15b_OMe_FL_PS_EC | 0.06 | 0.0039 |

### Example 2

### Inhibition of miR-15b In Vivo

Ten inhibitors (polynucleotides 5-14 from Table 5) targeting miR-15b were synthesized and tested in normal mice for the effect on miR-15b levels. The mice (n=4) were dosed 80mg/kg through a low pressure tail vein injection and tissues were analyzed four days later for miR-15b levels. Both the liver and the heart were analyzed and the data compared to saline injected mice.

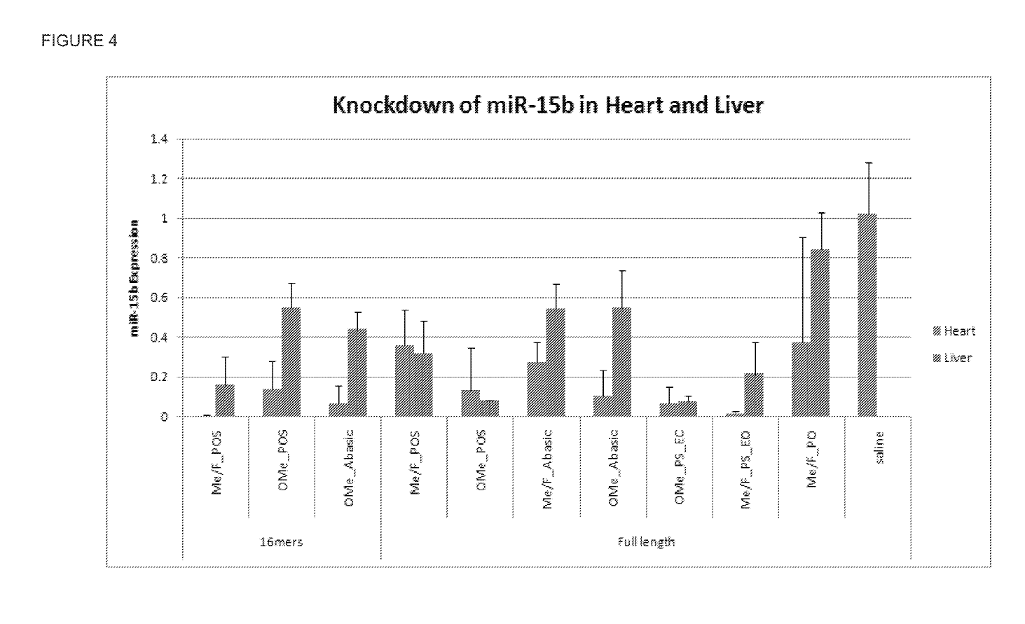

In both the liver and the heart, the inhibitors with the phosphorothioate monophosphate caps (POS) showed strong inhibition of miR-15b (See FIG. 4). This was quite surprising that these molecules without any internal phosphorothioate linkages or cholesterol conjugate were able to show such an effect in the heart.

These experiments demonstrate that there are unique modification motifs that enhance potency for miRNA inhibitors. Nuclease stability may be an important indicator as molecules that are entirely phosphodiester linkages with 2′OMe modifications are less effective than molecules with phosphorothioate linkages. The one exception seems to be when the ends are either capped with Abasic nucleosides or terminal phosphorothioate monophosphates. Even as a 16 mer, this end-capped molecule has an IC50 of 80 pM while the full length polynucleotide has an IC50 of 180 pM. This modification pattern: 2′OMe polynucleotide with terminal phosphorothioate monophosphates is a unique motif.

### Example 3

### Inhibition of miR-208a

The full length and 16-mer miR-208a inhibitors were prepared and tested in neonatal rat cardiomyocytes 48 hours post transfection b e expression of bMHC (determined by quantitative PCR). Inhibitors were tested at 100 nM and 1 nM.

Inhibitors tested included 2′ positions modified as either: all 2′OMe; A and G modified as 2′OMe, with C and U modified as 2′F; and deoxy A and G, with 2′OMe C and U. Cap structures included abasic and phosphorothioate monophosphate capped.

miR-208 is required for up-regulation of bMHC expression in response to cardiac stress and for repression of fast skeletal muscle genes in the heart. See WO 2009/018492 and 2008/016924, each of which are hereby incorporated by reference.

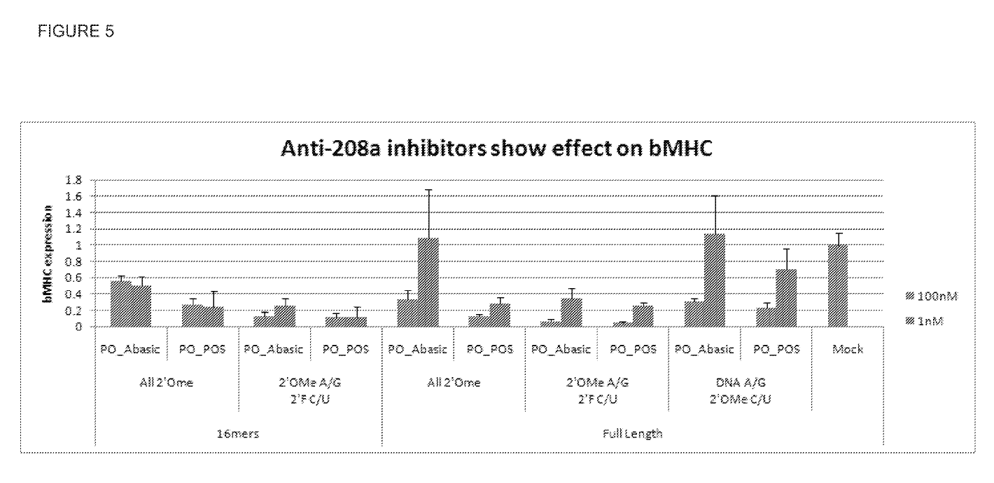

The results are shown in FIG. 5. As shown, 2′ modified polynucleotides with end caps were effective for inhibiting miR-208a, even at 1 nM concentration.

### Example 4

### Inhibition of miR-21

The miR-21 inhibitors (end-capped) were tested in vitro at 100 nM using the dual luciferase assay in HeLa cells. The results are shown in FIG. 6. As shown, inhibitors having all 2′OMe with abasic moiety end-caps or end-capped with phosphorothioate monophosphate were particularly effective.

The polynucleotides shown in the following Table 6 in relation to miR-15b, miR208, and miR-21 inhibitors, were synthesized.

TABLE 6

| miRNA | | | |
|---|---|---|---|
| target | Alias | Sequence (5′ to 3′) | Length |
| 15b | 15b_OMe_16_POS | ps-mAmCmCmAmUmGmAmUmGmUmGmCmUmGmCmU-ps | 16 |
| 15b | 15b_Me/F_16_POS | ps-mAfCfCmAfUmGmAfUmGfUmGfCfUmGfCfU-ps | 16 |
| 15b | 15b_OMe_FL_POS | ps-mUmGmUmAmAmAmCmCmAfUmGmAmUmGmUmGmCmUmGmCmUmA-ps | 22 |
| 15b | 15b_Me/F_FL_POS | ps-fUmGfUmAmAmAfCfCmAfUmGmAfUmGfUmGfCfUmGfCfUMA-ps | 22 |
| 208 | 208a_OMe_16_POS | ps-mCmUmUmUmUmGmCmUmCmGmUmCmUmUmA-ps | 16 |
| 208 | 208a_Me/F_16_POS | ps-fCfUfUfUfUfUmGfCfUfCmGfUfCfUfUmA-ps | 16 |
| 208 | 208a_OMe_FL_POS | ps-mAmCmAmAmGmCmUmUmUmUmUmGmCmUmCmGmUmCmUmUmAmU-ps | 22 |
| 208 | 208a_Me/F_FL_POS | ps-mAfCmAmAmGfCfUfUfUmGfCfUfCmGfUfCfUfUmAfU-ps | 22 |
| 21 | 21_OMe_16_POS | ps-mAmUmCmAmGmUmCmUmGmAmUmAmAmGmCmU-ps | 16 |
| 21 | 21_OMe/F_16_POS | ps-mAfUfCmAmGfUfCfUmGmAfUmAmAmGfCfU-ps | 16 |
| 21 | 21_OMe_FL_POS | ps-mUmCmAmAmCmAmUmCmAmGmUmCmUmGmAmUmAmAmGmCmUmA-ps | 22 |
| 21 | 21_MeILFLPOS | ps-mFfCmAmAfCmAmFfCmAmGmFfCmFmGmAmFmAmAmGfCmFmA-ps | 22 |

### Example 5

### Tissue Distribution of miR-15b Inhibitors In Vivo

Four inhibitors of miR-15b (Table 7) were synthesized and injected into mice to assess their tissue biodistribution. Mice were treated with human angiotensin II (Ang II) administered via osmotic pump which was implanted subcutaneously on the dorsal side. Seven days following Ang II treatment, the mice were dosed at either 1×0.33 mg/kg, 1×1 mg/kg, 1×3.3 mg/kg, 1×33 mg/kg or 3×0.33 mg/kg. The last dose indicates that the mice were dosed on 3 subsequent days at 0.33 mg/kg. The animals were sacrificed on day 4 and the tissues were processed for the biodistribution assay. The Ang H treatment was sustained during the dosing regimen.

Table 7 lists the sequence and particular modifications of each of the oligos used in this experiment. Compound 10134 was comprised of LNA and 2′ deoxy nucleotides and a full phosphorothioate backbone. Compound 10115 was comprised of 2′OMe modifications and a full phosphorothioate backbone. Compound 10623 was comprised of 2′OMe modifications, a full phosphorothioate backbone and 3′ and 5′ phosphorothioate monophosphate. Compound 10624 was comprised of 2′OMe modifications, alternating phosphorothioate and phosphodiester linkages and 3′ and 5′ phosphorothioate monophosphate.

TABLE 7

| Cmpd# | Alias | Sequence (5' to 3') | Length |
|---|---|---|---|
| 10134 | 15b_DNA_LNA_16_PS | lAs; dCs; dCs; lAs; lTs; dGs; lAs; lTs; dGs; lTs; lGs; dCs; dTs; lGs; | 16 |
| | | dCs; lT | |
| 10623 | 15b_OMe_16_PS_POS | ps; mAs; mCs; mCs; mAs; mUs; mGs; mAs; mUs; mGs; mUs; mGs; mCs; mUs; | 16 |
| | | mGs; mCs; mUs; p | |
| 10624 | 15b_OMe_16_PSEO_POS | ps; mAs; mC; mCs; mA; mUs; mG; mAs; mU; mGs; mU; mGs; mC; mUs; mG; mCs; | 16 |
| | | mUs; p | |
| 10116 | 15b_OMe_16_PS | mAs; mCs; mCs; mAs; mUs; mGs; mAs; mUs; mGs; mUs; mGs; mCs; mUs; mGs; | 16 |
| | | mCs; mU | |

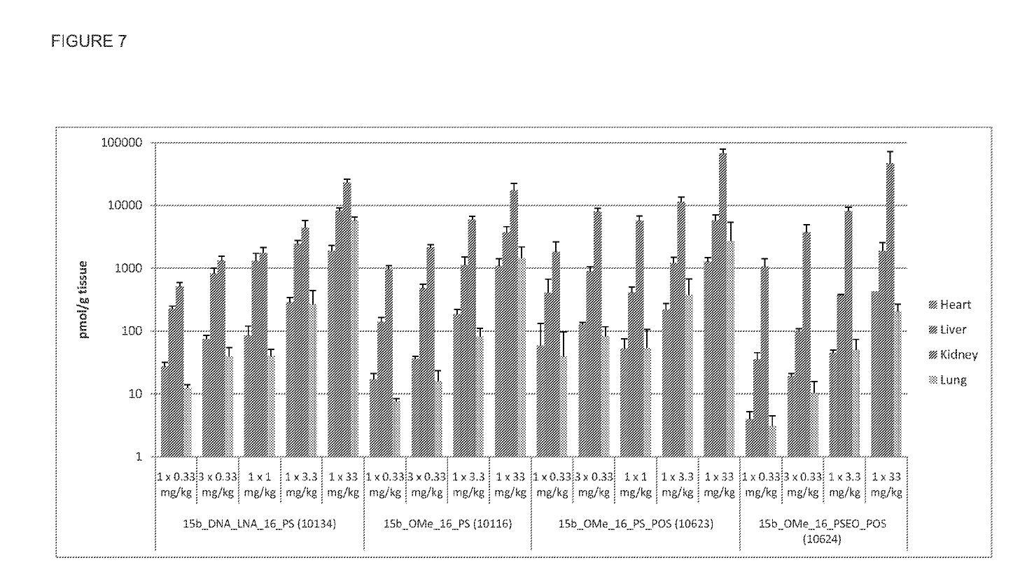

FIG. 7 shows accumulation of the inhibitors in the heart, liver, kidney, and lung. When comparing the capped vs. non-capped 2′OMe oligo, the amount of inhibitor delivered to all organs is often higher when capped with the POS modification. The effect is highest at the lowest dose of 1×0.33 mg/kg. Delivery to kidney remains fairly equivalent across all four modification patterns. The fully modified phosphorothioate backbone also shows higher delivery in heart, liver and lung compared to the every-other modification.

All publications, patents and patent applications discussed and cited herein are incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

The following references are hereby incorporated by reference in their entireties for all purposes.

B. Sproat et al., Nucleic Acids Research 17: 3373-3386 (1989).

E. L. Ruff et al., Journal of Organic Chemistry, 61: 1547-1550 (1996).

H. Cramer et al., Helvetica Chimica Acta, 79: 2114-2136 (1996).

Vermeulen A, et al., RNA 13:723-730 (2007).

U.S. Pat. No. 5,998,203

1-33. (canceled)

34. A polynucleotide consisting of about ten or less nucleotides, wherein the polynucleotide comprises an antisense sequence complementary to mature miR15b and is effective to inhibit miR15b, and wherein the polynucleotide comprises one or more internal phosphorothioate linkages and one or more locked nucleic acid residues.

35. The polynucleotide of claim 34, wherein the polynucleotide is at least about 75% complementary to mature miR15b.

36. The polynucleotide of claim 34, wherein said polynucleotide is about eight nucleotides or less and is 100% complementary to mature miR15b.

37. The polynucleotide of claim 34, wherein said polynucleotide is fully phosphorothioate-linked.

38. The polynucleotide of claim 34, wherein the polynucleotide comprises at least one terminal phosphorothioate monophosphate.

39. The polynucleotide of claim 34, wherein said polynucleotide consists of all locked nucleic acid residues.

40. The polynucleotide of claim 34, wherein the polynucleotide comprises the sequence 5′-GTGCTGCT-3′.

41. The polynucleotide of claim 34, wherein the polynucleotide consists of the sequence 5′-GTGCTGCT-3′, and the polynucleotide is fully phosphorothioate linked and consists of all locked nucleic acids.

42. A pharmaceutical composition comprising the polynucleotide of claim 1, and a pharmaceutically acceptable carrier.

43. The pharmaceutical composition of claim 42, wherein the composition is formulated as a colloidal dispersion system, macromolecular complex, nanocapsule, microsphere, bead, oil-in-water emulsion, micelle, mixed micelle, or liposome.

44. The pharmaceutical composition of claim 42, wherein the composition is formulated for intradermal delivery, subcutaneous delivery, intramuscular delivery, intraperitoneal or intravenous delivery.

45. The pharmaceutical composition of claim 42, wherein the composition is formulated for administration by a cardiac catheter system.

46. A method of treating a patient having a condition associated with miRNA expression, comprising administering the pharmaceutical composition of claim 42 to the patient.

47. The method of claim 46, wherein the condition is one or more of cardiac hypertrophy, myocardial infarction, heart failure, vascular damage, and pathologic cardiac fibrosis.

48. The method of claim 46, wherein the composition is administered by a cardiac catheter.

49. A polynucleotide having one or more nucleotide modifications at the 2′ position, and at least one terminal cap structure, wherein the polynucleotide comprises a miRNA or miRNA antisense nucleotide sequence.

50. The polynucleotide of claim 49, wherein the polynucleotide is from 5 to 25 nucleotides in length.

51. A pharmaceutical composition comprising the polynucleotide of claim 49, and a pharmaceutically acceptable carrier.

52. A method of treating a patient having a condition associated with miRNA expression, wherein the condition is one or more of cardiac hypertrophy, myocardial infarction, heart failure, vascular damage, and pathologic cardiac fibrosis, comprising administering the pharmaceutical composition of claim 51 to the patient.


SEQUENCE LISTING (122 sequences)

SEQ ID NO: 1 - RNA, 22 nt, Homo sapiens
uggaauguaaagaaguauguau

SEQ ID NO: 2 - RNA, 22 nt, Homo sapiens
aacccguagauccgaacuugug

SEQ ID NO: 3 - RNA, 23 nt, Homo sapiens
uacccuguagaaccgaauuugug

SEQ ID NO: 4 - RNA, 22 nt, Homo sapiens
ucccugagacccuaacuuguga

SEQ ID NO: 5 - RNA, 21 nt, Homo sapiens
ucacagugaaccggucucuuu

SEQ ID NO: 6 - RNA, 22 nt, Homo sapiens
uuugguccccuucaaccagcug

SEQ ID NO: 7 - RNA, 22 nt, Homo sapiens
uuugguccccuucaaccagcua

SEQ ID NO: 8 - RNA, 22 nt, Homo sapiens
ucuacagugcacgugucuccag

SEQ ID NO: 9 - RNA, 21 nt, Homo sapiens
ugagaugaagcacuguagcuc

SEQ ID NO: 10 - RNA, 23 nt, Homo sapiens
guccaguuuucccaggaaucccu

SEQ ID NO: 11 - RNA, 22 nt, Homo sapiens
ucucccaacccuuguaccagug

SEQ ID NO: 12 - RNA, 22 nt, Homo sapiens
uagcagcacauaaugguuugug

SEQ ID NO: 13 - RNA, 22 nt, Homo sapiens
uagcagcacaucaugguuuaca

SEQ ID NO: 14 - RNA, 22 nt, Homo sapiens
uagcagcacguaaauauuggcg

SEQ ID NO: 15 - RNA, 23 nt, Homo sapiens
aacauucauugcugucggugggu

SEQ ID NO: 16 - RNA, 21 nt, Homo sapiens
uagcagcacagaaauauuggc

SEQ ID NO: 17 - RNA, 22 nt, Homo sapiens
uucaccaccuucuccacccagc

SEQ ID NO: 18 - RNA, 23 nt, Homo sapiens
cccaguguucagacuaccuguuc

SEQ ID NO: 19 - RNA, 23 nt, Homo sapiens
cccaguguuuagacuaucuguuc

SEQ ID NO: 20 - RNA, 22 nt, Homo sapiens
acaguagucugcacauugguua

SEQ ID NO: 21 - RNA, 22 nt, Homo sapiens
uggaauguaaggaagugugugg

SEQ ID NO: 22 - RNA, 22 nt, Homo sapiens
auaagacgagcaaaaagcuugu

SEQ ID NO: 23 - RNA, 22 nt, Homo sapiens
auaagacgaacaaaagguuugu

SEQ ID NO: 24 - RNA, 23 nt, Homo sapiens
uaaagugcuuauagugcagguag

SEQ ID NO: 25 - RNA, 22 nt, Homo sapiens
uagcuuaucagacugauguuga

SEQ ID NO: 26 - RNA, 22 nt, Homo sapiens
acagcaggcacagacaggcagu

SEQ ID NO: 27 - RNA, 22 nt, Homo sapiens
aagcugccaguugaagaacugu

SEQ ID NO: 28 - RNA, 23 nt, Homo sapiens
agcuacauugucugcuggguuuc

SEQ ID NO: 29 - RNA, 21 nt, Homo sapiens
agcuacaucuggcuacugggu

SEQ ID NO: 30 - RNA, 21 nt, Homo sapiens
caagucacuagugguuccguu

SEQ ID NO: 31 - RNA, 21 nt, Homo sapiens
aucacauugccagggauuucc

SEQ ID NO: 32 - RNA, 22 nt, Homo sapiens
uucaaguaauccaggauaggcu

SEQ ID NO: 33 - RNA, 21 nt, Homo sapiens
uucaaguaauucaggauaggu

SEQ ID NO: 34 - RNA, 22 nt, Homo sapiens
aaggagcucacagucuauugag

SEQ ID NO: 35 - RNA, 22 nt, Homo sapiens
uagcaccaucugaaaucgguua

SEQ ID NO: 36 - RNA, 23 nt, Homo sapiens
uagcaccauuugaaaucaguguu

SEQ ID NO: 37 - RNA, 22 nt, Homo sapiens
uagcaccauuugaaaucgguua

SEQ ID NO: 38 - RNA, 22 nt, Homo sapiens
uguaaacauccucgacuggaag

SEQ ID NO: 39 - RNA, 22 nt, Homo sapiens
uguaaacauccuacacucagcu

SEQ ID NO: 40 - RNA, 23 nt, Homo sapiens
uguaaacauccuacacucucagc

SEQ ID NO: 41 - RNA, 22 nt, Homo sapiens
uguaaacauccccgacuggaag

SEQ ID NO: 42 - RNA, 22 nt, Homo sapiens
uguaaacauccuugacuggaag

SEQ ID NO: 43 - RNA, 23 nt, Homo sapiens
ucucacacagaaaucgcacccgu

SEQ ID NO: 44 - RNA, 22 nt, Homo sapiens
gaaguuguucgugguggauucg

SEQ ID NO: 45 - RNA, 22 nt, Homo sapiens
acuggacuuagggucagaaggc

SEQ ID NO: 46 - RNA, 21 nt, Homo sapiens
acuggacuuggagucagaagg

SEQ ID NO: 47 - RNA, 22 nt, Homo sapiens
cagcagcaauucauguuuugaa

SEQ ID NO: 48 - RNA, 21 nt, Homo sapiens
ucacuccucuccucccgucuu

SEQ ID NO: 49 - RNA, 22 nt, Homo sapiens
ucaggcucaguccccucccgau

SEQ ID NO: 50 - RNA, 22 nt, Homo sapiens
uccuguacugagcugccccgag

SEQ ID NO: 51 - RNA, 21 nt, Homo sapiens
cagcagcacacugugguuugu

SEQ ID NO: 52 - RNA, 21 nt, Homo sapiens
uuaagacuugcagugauguuu

SEQ ID NO: 53 - RNA, 23 nt, Homo sapiens
ucggggaucaucaugucacgaga

SEQ ID NO: 54 - RNA, 22 nt, Homo sapiens
uauugcacuugucccggccugu

SEQ ID NO: 55 - RNA, 22 nt, Homo sapiens
uauugcacucgucccggccucc

SEQ ID NO: 56 - RNA, 22 nt, Homo sapiens
ugagguaguagguuguauaguu

SEQ ID NO: 57 - RNA, 22 nt, Homo sapiens
ugagguaguagguugugugguu

SEQ ID NO: 58 - RNA, 22 nt, Homo sapiens
ugagguaguagguuguaugguu

SEQ ID NO: 59 - RNA, 22 nt, Homo sapiens
agagguaguagguugcauaguu

SEQ ID NO: 60 - RNA, 22 nt, Homo sapiens
ugagguaggagguuguauaguu

SEQ ID NO: 61 - RNA, 22 nt, Homo sapiens
ugagguaguagauuguauaguu

SEQ ID NO: 62 - RNA, 22 nt, Homo sapiens
ugagguaguaguuuguacaguu

SEQ ID NO: 63 - RNA, 22 nt, Homo sapiens
aaaccguuaccauuacugaguu

SEQ ID NO: 64 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 65 - DNA, 71 bp, Homo sapiens
acgggcgagcuuuuggcccggguuauaccugaugcucacguauaagacgagcaaaaagcutguuggucaga

SEQ ID NO: 66 - RNA, 72 nt, Homo sapiens
ugucggguagcuuaucagacugauguugacuguugaaucucauggcaacaccagucgaugggcugucugaca

SEQ ID NO: 67 - RNA, 16 nt, Artificial Sequence
  Anti-miR-15b polynucleotide
accaugaugugcugcu

SEQ ID NO: 68 - RNA, 16 nt, Artificial Sequence
  Anti-miR-15b polynucleotide
accaugaugugcugcu

SEQ ID NO: 69 - RNA, 22 nt, Artificial Sequence
  misc_feature (1)..(1): May have a phosphorothioate monophosphate cap
uguaaaccaugaugugcugcua

SEQ ID NO: 70 - RNA, 22 nt, Artificial Sequence
  Anti-miR-15b polynucleotide
uguaaaccaugaugugcugcua

SEQ ID NO: 71 - RNA, 16 nt, Artificial Sequence
  Anti-miR-208a polynucleotide
cuuuuugcucgucuua

SEQ ID NO: 72 - RNA, 16 nt, Artificial Sequence
  Anti-miR-208a polynucleotide
cuuuuugcucgucuua

SEQ ID NO: 73 - RNA, 22 nt, Artificial Sequence
  Anti-miR-208a polynucleotide
acaagcuuuuugcucgucuuau

SEQ ID NO: 74 - RNA, 22 nt, Artificial Sequence
  Anti-miR-208a polynucleotide
acaagcuuuuugcucgucuuau

SEQ ID NO: 75 - RNA, 16 nt, Artificial Sequence
  Anti-miR-21 polynucleotide
aucagucugauaagcu

SEQ ID NO: 76 - RNA, 16 nt, Artificial Sequence
  Anti-miR-21 polynucleotide
aucagucugauaagcu

SEQ ID NO: 77 - RNA, 22 nt, Artificial Sequence
  Anti-miR-21 polynucleotide
ucaacaucagucugauaagcua

SEQ ID NO: 78 - RNA, 22 nt, Artificial Sequence
  Anti-miR-21 polynucleotide
ucaacaucagucugauaagcua

SEQ ID NO: 79 - RNA, 16 nt, Artificial Sequence
  Anti-miR-15b polynucleotide
accaugaugugcugcu

SEQ ID NO: 80 - RNA, 16 nt, Artificial Sequence
  Anti-miR-15b polynucleotide
accaugaugugcugcu

SEQ ID NO: 81 - RNA, 16 nt, Artificial Sequence
  Anti-miR-15b polynucleotide
accaugaugugcugcu

SEQ ID NO: 82 - RNA, 16 nt, Artificial Sequence
  Anti-miR-15b polynucleotide
accaugaugugcugcu

SEQ ID NO: 83 - RNA, 16 nt, Artificial Sequence
  Anti-miR-15b polynucleotide
accaugaugugcugcu

SEQ ID NO: 84 - RNA, 16 nt, Artificial Sequence
  Anti-miR-15b polynucleotide
accaugaugugcugcu

SEQ ID NO: 85 - RNA, 16 nt, Artificial Sequence
  Anti-miR-15b polynucleotide
accaugaugugcugcu

SEQ ID NO: 86 - RNA, 16 nt, Artificial Sequence
  Anti-miR-15b polynucleotide
accaugaugugcugcu

SEQ ID NO: 87 - RNA, 16 nt, Artificial Sequence
  Anti-miR-15b polynucleotide
accaugaugugcugcu

SEQ ID NO: 88 - RNA, 16 nt, Artificial Sequence
  Anti-miR-15b polynucleotide
accaugaugugcugcu

SEQ ID NO: 89 - RNA, 16 nt, Artificial Sequence
  Anti-miR-15b polynucleotide
accaugaugugcugcu

SEQ ID NO: 90 - RNA, 16 nt, Artificial Sequence
  Anti-miR-15b polynucleotide
accaugaugugcugcu

SEQ ID NO: 91 - RNA, 16 nt, Artificial Sequence
  Anti-miR-15b polynucleotide
accaugaugugcugcu

SEQ ID NO: 92 - RNA, 16 nt, Artificial Sequence
  Anti-miR-15b polynucleotide
accaugaugugcugcu

SEQ ID NO: 93 - RNA, 16 nt, Artificial Sequence
  Anti-miR-15b polynucleotide
accaugaugugcugcu

SEQ ID NO: 94 - RNA, 16 nt, Artificial Sequence
  Anti-miR-15b polynucleotide
accaugaugugcugcu

SEQ ID NO: 95 - RNA, 16 nt, Artificial Sequence
  Anti-miR-15b polynucleotide
accaugaugugcugcu

SEQ ID NO: 96 - RNA, 16 nt, Artificial Sequence
  Anti-miR-15b polynucleotide
accaugaugugcugcu

SEQ ID NO: 97 - DNA, 16 bp, Artificial Sequence
  Anti-miR-15b polynucleotide
accatgatgtgctgct

SEQ ID NO: 98 - DNA, 16 bp, Artificial Sequence
  Anti-miR-15b polynucleotide
accatgatgtgctgct

SEQ ID NO: 99 - DNA, 16 bp, Artificial Sequence
  Anti-miR-15b polynucleotide
accatgatgtgctgct

SEQ ID NO: 100 - RNA, 22 nt, Artificial Sequence
  Anti-miR-15b polynucleotide
uguaaaccaugaugugcugcua

SEQ ID NO: 101 - RNA, 22 nt, Artificial Sequence
  Anti-miR-15b polynucleotide
uguaaaccaugaugugcugcua

SEQ ID NO: 102 - RNA, 22 nt, Artificial Sequence
  Anti-miR-15b polynucleotide
uguaaaccaugaugugcugcua

SEQ ID NO: 103 - RNA, 22 nt, Artificial Sequence
  Anti-miR-15b polynucleotide
uguaaaccaugaugugcugcua

SEQ ID NO: 104 - RNA, 22 nt, Artificial Sequence
  Anti-miR-15b polynucleotide
uguaaaccaugaugugcugcua

SEQ ID NO: 105 - RNA, 22 nt, Artificial Sequence
  Anti-miR-15b polynucleotide
uguaaaccaugaugugcugcua

SEQ ID NO: 106 - RNA, 22 nt, Artificial Sequence
  Anti-miR-15b polynucleotide
uguaaaccaugaugugcugcua

SEQ ID NO: 107 - RNA, 22 nt, Artificial Sequence
  Anti-miR-15b polynucleotide
uguaaaccaugaugugcugcua

SEQ ID NO: 108 - RNA, 22 nt, Artificial Sequence
  Anti-miR-15b polynucleotide
uguaaaccaugaugugcugcua

SEQ ID NO: 109 - RNA, 22 nt, Artificial Sequence
  Anti-miR-15b polynucleotide
uguaaaccaugaugugcugcua

SEQ ID NO: 110 - RNA, 22 nt, Artificial Sequence
  Anti-miR-15b polynucleotide
uguaaaccaugaugugcugcua

SEQ ID NO: 111 - RNA, 22 nt, Artificial Sequence
  Anti-miR-15b polynucleotide
uguaaaccaugaugugcugcua

SEQ ID NO: 112 - RNA, 22 nt, Artificial Sequence
  Anti-miR-15b polynucleotide
uguaaaccaugaugugcugcua

SEQ ID NO: 113 - RNA, 22 nt, Artificial Sequence
  Anti-miR-15b polynucleotide
uguaaaccaugaugugcugcua

SEQ ID NO: 114 - RNA, 22 nt, Artificial Sequence
  Anti-miR-15b polynucleotide
uguaaaccaugaugugcugcua

SEQ ID NO: 115 - RNA, 22 nt, Artificial Sequence
  Anti-miR-15b polynucleotide
uguaaaccaugaugugcugcua

SEQ ID NO: 116 - RNA, 22 nt, Artificial Sequence
  Anti-miR-15b polynucleotide
uguaaaccaugaugugcugcua

SEQ ID NO: 117 - RNA, 22 nt, Artificial Sequence
  Anti-miR-15b polynucleotide
uguaaaccaugaugugcugcua

SEQ ID NO: 118 - DNA, 22 bp, Artificial Sequence
  Anti-miR-15b polynucleotide
tgtaaaccatgatgtgctgcta

SEQ ID NO: 119 - DNA, 22 bp, Artificial Sequence
  Anti-miR-15b polynucleotide
tgtaaaccatgatgtgctgcta

SEQ ID NO: 120 - DNA, 22 bp, Artificial Sequence
  Anti-miR-15b polynucleotide
tgtaaaccatgatgtgctgcta

SEQ ID NO: 121 - RNA, 16 nt, Artificial Sequence
  Anti-miR-15b polynucleotide
accaugaugugcugcu

SEQ ID NO: 122 - RNA, 16 nt, Artificial Sequence
  Anti-miR-15b polynucleotide
accaugaugugcugcu